(19) United States
(12) Patent Application Publication
(10) Pub. No.: US 2009/0030290 A1
(43) Pub. Date: Jan. 29, 2009

(54) METHOD AND APPARATUS FOR AUTOMATED DIFFERENTIATED DIAGNOSIS OF ILLNESS

(76) Inventors: Michael J. Kozuch, Pleasanton, CA (US); James E. Smith, III, Bush, LA (US); Timothy A. Fate, San Ramon, CA (US)

Correspondence Address: DAVID S. NAGY, 756 S. GRIFFITH PARK DR., BURBANK, CA, 91506, US

(21) Appl. No.: 12/172,419

(22) Filed: Jul. 14, 2008

Related U.S. Application Data
(60) Provisional application No. 60/951,418, filed on Jul. 23, 2007.

(51) Int. Cl. A61B 5/02; A61B 5/00; G06Q 50/00

(52) U.S. Cl. 600/301; 600/300; 705/2

(57) ABSTRACT

This method and apparatus for automated differential diagnosis of illness utilizes neural network technology to analyze information that has been collected and assimilated from multiple sources, including information concerning lifestyle and travel habits, occupational and environmental risks and other contributory factors, and compares this information to, and incorporates it into, databases, to render diagnoses as well as alerts regarding anomalous concentrations of illnesses. The invention has similar application in the area of mechanical maintenance and repair.

This application is the non-provisional version of provisional application 60/951,418 of the same name and by the same inventors, filed on Jul. 23, 2007, and the disclosure of that earlier application is hereby incorporated by reference.

### BACKGROUND OF THE INVENTION

1. Field of Invention

The present invention relates to the field of medical diagnosis, and particularly risks such as disease or illness. The invention is more particularly related to a comprehensive statistical analysis of past and medical factors and other external conditions that allow an accurate differentiated diagnosis of disease or illness.

2. Discussion of Background

The ability to quickly and accurately diagnose symptoms is becoming increasingly necessary to minimize the cost of extended patient care, limit medical liabilities, and deal with the enormous complexity of medical conditions due to environmental risk factors. There are numerous hospital institutions that are moving to digitize patient medical records, however this has proven to be a slow and expensive process to implement.

The cost of preventable medical errors is now approximately $29 billion per year and continues to escalate. This cost to society is reflected in the loss of productivity, the cost of extended medical care, and the cost of drugs and treatments (that may or may not be appropriate). This is just the cost of preventable errors, not including the cost of malpractice.

With the onset of fatigue, increasing numbers of patients seen per day, the enormous volume and complexity of medical information, and the threat of malpractice, doctors are under severe stress to perform well. Rapid and accurate diagnosis of illness would be invaluable to doctors, to get patients into the right treatment as soon as possible.

There have been numerous attempts to accurately diagnose illnesses, but most systems offer too many diagnostic possibilities, offer inaccurate results, or draw on a limited number of diseases from which to draw conclusions. The auto-diagnostic systems most commonly used rely on medical encyclopedias or databases that contain the average or predominant symptoms associated with illnesses.

There have been attempts to use automated systems to diagnose illness. Internist-1 was an artificial intelligent program that was designed for this purpose but was cumbersome and yielded mixed results. Some of the newer developments use branching tree structures or relational databases in their logic, but these systems are very large, rigid and are not able to cope with symptoms that might be common to multiple illnesses. Neural networks are just now beginning to emerge in radiological diagnosis and interpretation. Neural networks and systems that reflect adaptive learning have been used in a multitude of engineering applications and are slowly working their way into medical practice.

As the genome project gathers momentum in mapping the human genetic code there will greater emphasis placed on identifying a patient's predisposition to certain illnesses based on his or her genetic makeup. While this information will be useful in identifying the risk of illness, there is no actual guarantee that the individual will develop that condition.

Factors that contribute to disease depend upon the exposure of an individual to biological, chemical, electromagnetic or any kind of adverse stress factors that interact with their physiology to bring about illness or changes in physiology or chemistry. Therefore, our approach looks to identify all the principal factors and patterns that might trigger a physical reaction leading to an illness or anomalous physiological behavior. While there are numerous studies identifying a plethora of these triggers, there is no integrated platform that incorporates all the environmental risk factors to which an individual might have been exposed during his or her lifetime. When an individual is unable to adapt to changes in their environment, then the landscape of stress conditions will trigger adverse physiological reactions leading to illness. In the extreme case, biological systems that refuse to change with the environment eventually lead to extinction.

### SUMMARY OF THE INVENTION

We have invented a new technique and apparatus that correlates and recognizes patterns in symptoms associated with environmental risk factors specific to individuals, their medical history, their family histories of illness, and a plethora of additional parameters to allow meaningful and accurate diagnosis of illnesses, diseases or any medical conditions that require treatment. The present invention includes the design of an integrated system of components that provide a new approach and methodology to diagnose symptoms of illness in order to enable doctors and medical practitioners to quickly diagnose medical conditions and ensure proper and immediate treatment. The system also provides alerts when a series of symptoms emerge multiple individuals in the database or reach a rate of change that might reflect the outbreak of an illness or require immediate attention. There is also immense predictive value in the invention in that treatments can also be recommended based on patterns of prior treatments.

In one embodiment, the present invention provides a differentiated diagnostic process that integrates diverse data sets in conjunction with current patient patterns in order to estimate a future state. Although the present invention focuses on patient illnesses, from a broader perspective, the occurrence of any event or risk can be modeled using similar processes so long as sufficient diverse datasets related to the event or risk are available.

Portions of both the device and method may be conveniently implemented in programming on a general purpose computer, or networked computers, and the results may be displayed on an output device connected to any of the general purpose, networked computers, or transmitted to a remote device for output or display. In addition, any components of the present invention represented in a computer program, data sequences, and/or control signals may be embodied as an electronic signal broadcast (or transmitted) at any frequency in any medium including, but not limited to, wireless broadcasts, and transmissions over copper wire(s), fiber optic cable(s), and co-ax cable(s), etc.

### BRIEF DESCRIPTION OF THE DRAWINGS

A more complete appreciation of the invention will be readily obtained by reference to the following detailed description when considered in connection with the accompanying drawings, wherein:

FIG. 1 is a high level flowchart of an automated diagnostic system according to an embodiment of the present invention;

FIG. 2 is a flow diagram of the comprehensive data entry by the patient into both electronic medical records as well as into the system that will lead to diagnosis according to an embodiment of the present invention;

FIG. 3 is a flow diagram of the vital signs data entry by the nurse into both electronic medical records as well as into the system that will lead to diagnosis according to an embodiment of the present invention;

FIG. 4 is a flow diagram of the data entry and link to the diagnostic tool by the doctor into both electronic medical records as well as into the system that will lead to diagnosis according to an embodiment of the present invention;

FIG. 5 is a flow diagram illustrating the flow of communication between the AI software and databases and users according to an embodiment of the present invention;

FIG. 6 is a flow diagram illustrating the alert system according to an embodiment of the present invention;

FIG. 7 is a drawing illustrating the clustering association between different symptoms due to the presence of different risk factors according to an embodiment of the present invention;

FIG. 8 is a drawing illustrating the conceptual configuration of multiple datasets used in a neural network according to an embodiment of the present invention;

### DESCRIPTION OF THE PREFERRED EMBODIMENTS

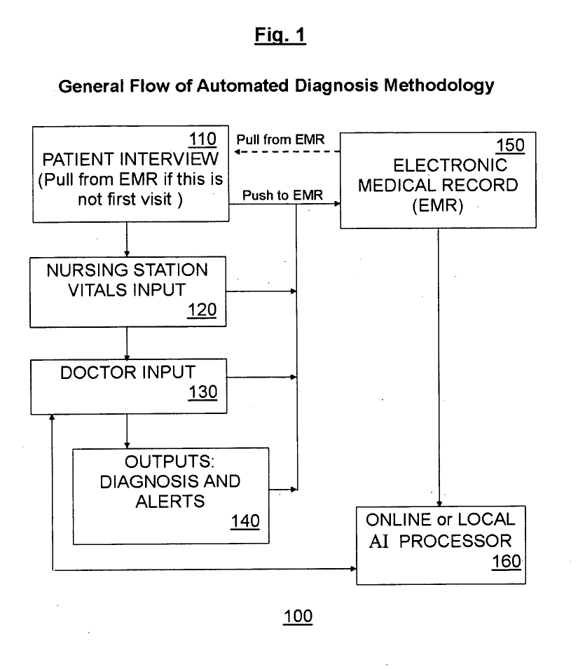

Referring now to the drawings, wherein like reference numerals designate identical or corresponding parts, and more particularly to FIG. 1 thereof, there is illustrated an overview of a diagnostic process (100) which includes the collection of patient inputs (110), a series of nurse inputs (120), a series of doctor inputs (130) and diagnostic outputs, alerts and treatments (140), a series of pattern recognition computations (neural network(s)) that occur either locally or remotely (160). Each of the input steps also feeds into a hospital's electronic medical records (150).

While the diagnosis of the potential of a particular illness is established using statistical pattern recognition techniques inherent in the neural network, there is also a physical basis for changes in the potential of an illness. The human body can be thought of existing in a metastable state and the presence of a risk factor acts to increase the stress on that point thereby causing a mutation, lapse in physiological function or illness. Should multiple risk factors be present at the same location and point in time, then the probability of an illness or symptom is increased. This predictive value of the invention can also be used in recommending treatment for particular illnesses where treatments under different environmental conditions yield different results. For example, a patient who normally would require penicillin as a treatment for a specific illness might be allergic to this drug. In this case the allergy represents the environmental risk factor, and so another drug would be recommended. Many other more complex examples exist with more obscure risk factors. The integration of a multitude of databases and environmental risk factors onto a common platform can also be used in the screening of individuals for clinical studies as well as the real-time monitoring of the use of pharmaceuticals in the marketplace.

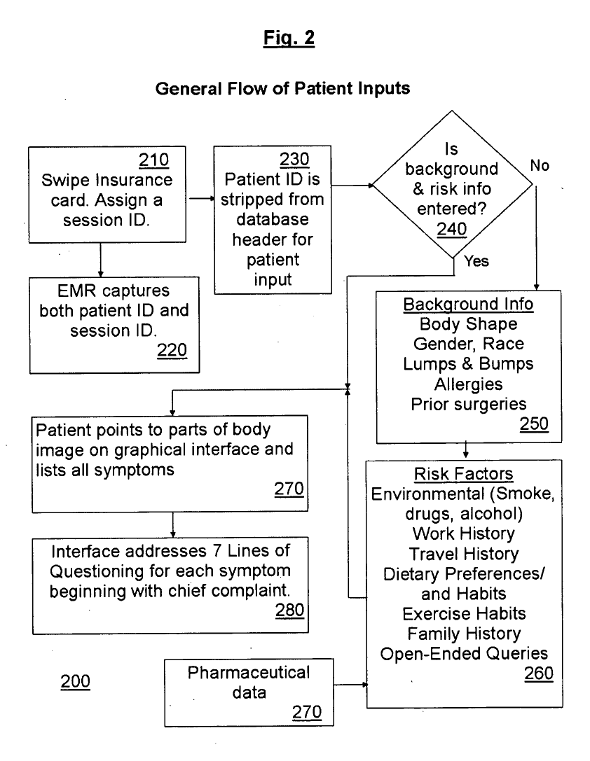

FIG. 2 illustrates an example process (200) for the acquisition of diverse data directly from the patient. Initially, a patient's insurance or identification number is input into a hospital register which also assigns a “session ID”. At this stage, all the patient inputs will eventually be entered into both the hospital's electronic medical records (EMR) as well as into the present software which resides either online or in a portable electronic device.

The EMR will capture the patient identification number as well as the session ID (220) and eventually all the portable electronic device inputs will be captured by the EMR as well. The hand held device, however will only carry the session number (230) to minimize any security risk if the portable device is lost. Only will the hospital EMR will contain the link between the session and the patient identity (name, address, and other related personal information). The advantage of a hand held portable device is that rapid response applications are possible such as in ambulances where paramedics can enter critical information into an electronic format prior to arrival at the hospital.

If the patient has never entered the data before or wishes to update prior records (240), then a process (250) will commence with a series of questions addressing the patient's background in terms of their body size and shape, allergies, vaccination history, medical surgeries and treatments (current or past), and any other marks, lumps or features that appear anomalous. Following this line of questioning, a new process (260) will then commence where the patient is asked a series of questions that address their environmental risk factors including their family medical history (e.g., is heart disease prevalent in the family?), social habits (including drinking, drug abuse, and sexual activity), places of travel or residence, dietary habits, exercise regimen, employment type, and any other information relevant to their lifestyle. This step will also query which medications or treatments they are undergoing. If the patient is not familiar with the names of specific medications, then they would be asked for the pharmacy that might have their name on record so that an automatic request can be made directly to the pharmacy for information on the patient's prescription drug purchase or use.

Finally the patient is asked to identify her symptoms according to the example process (270), beginning with her chief symptom and indicating any other symptom by pointing to a graphic of the human body on a touch screen monitor. Once each complaint or symptom location is logged, then each symptom is run through the standard seven lines of medical questioning (280). These lines of questioning include the quality of symptom, severity, quantity, timing, setting, things that make it better or worse, and finally the associated symptoms.

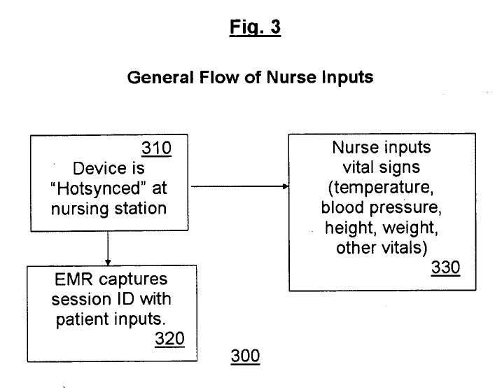

FIG. 3 illustrates process (300) of how the patient then allows the nursing station to “hot sync” the information (310) from the portable device or workstation into the electronic medical record (320). Since the EMR already has the patient and session identification numbers, it can then tag any input to a specific patient. The nurse then takes vital signs of the patient (330) such as temperature, blood pressure, body weight, and any other measurements and enters them into the portable electronic device or to the online site prior to visiting the doctor.

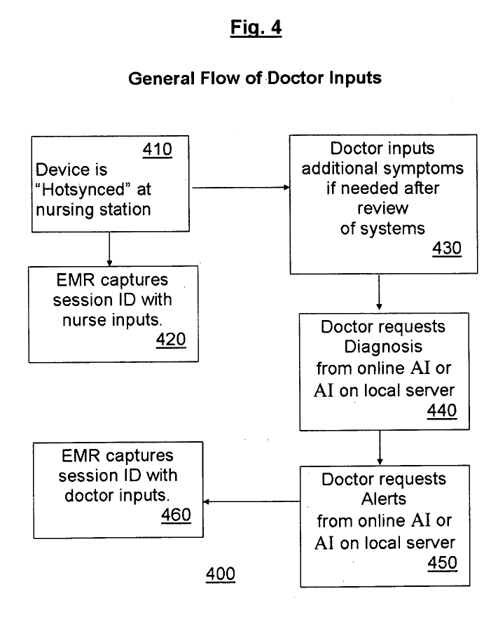

FIG. 4 illustrates the process (400) of how a doctor will enter additional symptomatic information and request an automated diagnosis. The electronic device or website account is “hot synced” to the EMR (410) so that the EMR captures all the vitals information (420). The device or website then displays all the gathered information to date which allows to the doctor to further review organ systems or ask more specific questions about any of the symptoms (430). Once the doctor is satisfied that he or she has all the required information, he or she requests an automated diagnosis (440) from the artificial intelligence software. The diagnosis will be a short report or listing of an illness or series of illnesses. Each illness might also show an associated probability of accuracy if requested. The computation of diagnosis is carried out on the local server or in-house computer. The doctor also has the option of requesting an alert (450), which will indicate whether the diagnosed illness(es) correspond to any contagious illness or epidemic outbreaks. At the end of the session, the portable device is “hot synced” with the hospital's EMR (460) where the patient information will reside.

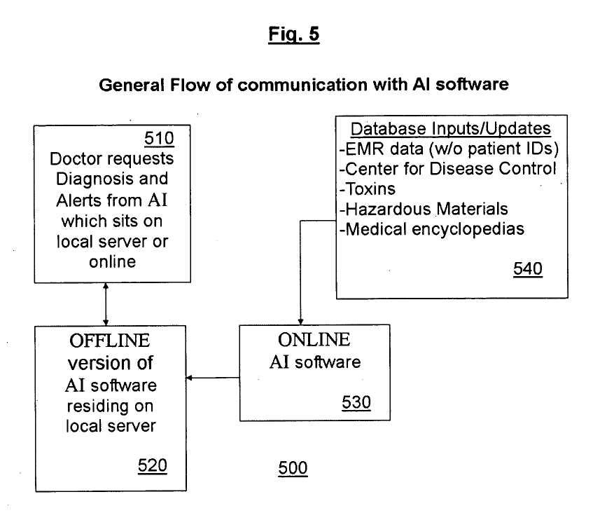

The core artificial intelligent software will reside on a computer in a central location from which remote sites such as hospitals or online access points will access the software for updates and computation as illustrated in the example process (500) in FIG. 5. When a doctor makes a request for a diagnosis (510) an electronic signal will trigger the initiation of the artificial intelligent computation from a computer that resides locally in the hospital (520). At regular intervals the local computer or server will be updated from the central location through the Internet (530). The artificial intelligence software at the central location will also be receiving updates from the various databases (540) which are used in the pattern recognition algorithm employed by the artificial intelligence software. These databases include any or all of EMR data (without patient ID numbers), the Center for Disease Control list of diseases and symptoms, toxins and their symptoms, hazardous materials and their symptoms, medical encyclopedias, and similar databases that reflect medical illness and associated symptoms.

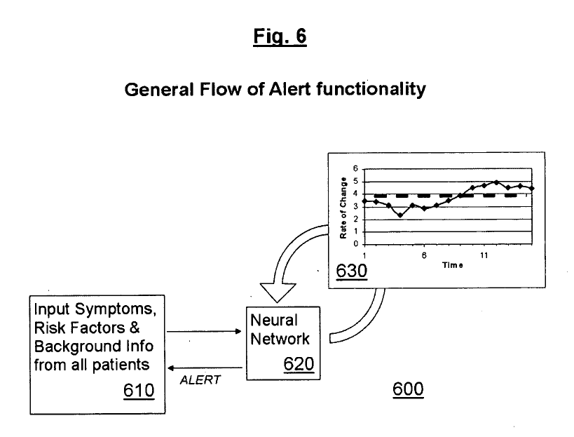

FIG. 6 illustrates an example of the alert process (600) in which all the patient information regarding background medical history and symptoms (610) are input to the neural network (620). As the neural network computes the relationship between the new data and all the inputs from other patients in the system it looks for similar patterns of symptoms or illness and the rate of increase (or change) of these patterns (630). Once these symptoms reach a threshold rate of change, which usually indicates how fast the symptom is spreading, an alert is sent to all hospitals in the system. Alternatively, if an individual is entering their symptomatic information remotely (e.g. at home) prior to an appointed admission and the neural network decides that a certain threshold of criticality is reached, then an alert will be sent to the patient to immediately come in to a hospital for treatment.

The alert process can also be extended beyond the recognition of changes in symptoms to include changes in environmental risk factors beyond the control of the patient. For example, should the Center for Disease control issue a warning about the spread of a new disease or if a natural hazard occurs which is known to affect human physiology, then this notice can readily be accepted by the neural network since the alert weights are adjusted continually. As an example it is known that geomagnetic storms affect melatonin levels in humans which in turn can be correlated (in part) to epileptic seizures or the number of hospital admissions with symptoms of depression. Therefore, patients who are already at risk from these conditions might be at a greater risk when there is a geomagnetic storm. An alert would allow physicians to design treatments to deal with these different circumstances.

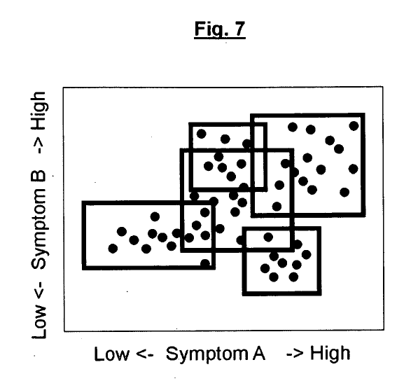

FIG. 7 illustrates the process of how symptoms might be related to other symptoms. Alternatively, symptoms can be plotted against other environmental risk factors. It is expected that there will most likely be groupings or clusters that emerge in these plots. The dimensions and spread of each cluster is due to other environmental risk factors that might influence the plotted symptoms to a greater or lesser degree. The artificial intelligence software maps out these variations and is therefore able to take subtle variations and determine weights of influence to determine the final diagnosis. It is expected that there may be several overlapping clusters and therefore there can be several weighted combinations of symptoms and environmental risk factors that influence the final diagnostic outcome.

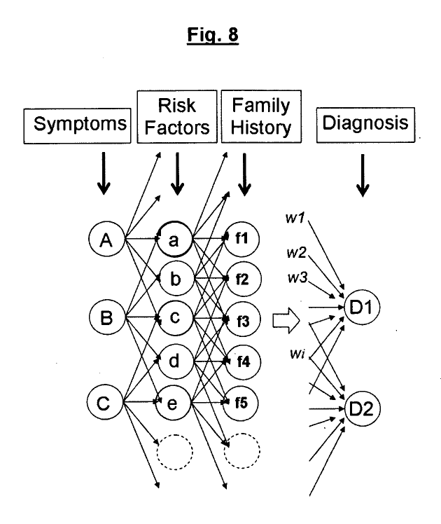

Neural networks are a common form of artificial intelligence software that is used to simulate the complex structure of neural connections in the brain or living organisms. FIG. 8 illustrates how a specific outcome (or disease) is the result of a multitude of interconnected processes (800). The weights of each of the neural connections are equivalent to the weights of overlapping symptoms that were shown in FIG. 7. In addition, there may be additional hidden layers of interconnected processes that are typical of computational neural network algorithms.

As an alternative to determining formal weights, one might solely log the presence and intensity of any of the symptoms. This log can then be called upon as a pattern recognition tool to be used in the diagnostic process. For example, if three different symptoms are present at a similar time and location, then future simultaneous occurrences of those symptoms would imply that the same illness might be present in other patients or could be forthcoming in areas where similar anomalous risk factors are present.

These data-driven rules can also be improved upon by adopting neurofuzzy algorithms (e.g., fuzzy logic) which can yield significantly more rules to include nearly all of the data parameters such as in FIG. 7. These algorithms incorporate the benefits of both neural networks and fuzzy logic with the following features:
the system is able to dynamically extract knowledge from the data to “learn” and rapidly improve its performance over time
weights can be readily adjusted continuously
fuzzy models can explain very complex systems with simple rules
qualitative (e.g. linguistic) and quantitative information can be combined

Neurofuzzy algorithms have the ability to calculate all the correlations and assign trigger weights to all the data types simultaneously. Neurofuzzy algorithms have been widely used by control engineers in designing video cameras, controlling subway systems, flight control, etc. A key feature of neurocontrol is that these systems lend themselves to control systems whose dynamics are highly nonlinear and unknown or uncertain. Other examples can be found in neurofuzzy systems that control wheel wear, obstacle avoidance behavior of mobile robots, image processing, and the control of carbon monoxide levels at traffic intersections in Japan.

The present invention includes a computer program product which is a storage medium (media) having instructions stored thereon/in which can be used to control, or cause, a computer to perform any of the processes of the present invention. The storage medium can include, but is not limited to, any type of disk including floppy disks, mini disks (MD's), optical discs, DVD, CD-ROMS, CDRW±, micro-drive, and magneto-optical disks, ROMs, RAMs, EPROMs, EEPROMs, DRAMs, VRAMs, flash memory devices (including flash cards, memory sticks), magnetic or optical cards, MEMS, nanosystems (including molecular memory ICs), RAID devices, remote data storage/archive/warehousing, or any type of media or device suitable for storing instructions and/or data.

Stored on any one of the computer readable media, the present invention includes software for controlling both the hardware of the general purpose/specialized computer or microprocessor, and for enabling the computer or microprocessor to interact with a human user or other mechanism utilizing the results of the present invention. Such software may include, but is not limited to, device drivers, operating systems, and user applications. Ultimately, such computer readable media further includes software for performing the present invention, as described above.

Included in the programming (software) of the general/specialized computer or microprocessor are software modules for implementing the teachings of the present invention, including, but not limited to, collecting medical data, correlating medical data, establishing triggers and weights, broadcasting alerts based on correlated data, triggers, and weights, and applying alerts to emergency management and other markets, and the display, storage, and/or communication of results according to the processes of the present invention.

The present invention may suitably comprise, consist of, or consist essentially of, any element of the various parts or features of the invention, and their equivalents as described herein. Further, the present invention illustratively disclosed herein may be practiced in the absence of any element, whether or not specifically disclosed herein. Obviously, numerous modifications and variations of the present invention are possible in light of the above teachings. It is therefore to be understood that within the scope of the appended claims, the invention may be practiced otherwise than as specifically described herein.

Plainly, this invention is as applicable to the treatment of animals as humans. While this invention was conceived to address pressing needs in the medical field, it can be seen that it is readily adaptable to other arenas in which entities require “healing”, such as automobiles or airplanes, or machinery of any sort. Mechanics and repair individuals (the analogs to medical professionals) would collect information and “vital signs” in much the same way, enter the information into the system, which would have access to analogous databases and which would supply and be supplied information concerning alerts (in this arena “epidemics” would equate to the discovery of disproportionate numbers of faulty or failing parts, and the alerts would lead to recalls). Thus, while the descriptions herein have focused on medical applications, they can all be seen to have their analogs in other applications

We claim:

1. A method for automated differentiated diagnosis of anomalies in a subject, comprising:
acquiring information and indications about the subject—including the subject's individual, family and medical or other remedial history, plus physical, environmental, occupational and lifestyle information—through interview and/or accessing subject records resident in a computer system, which computer system also incorporates and/or accesses databases on the familial, environmental and occupational factors relating to anomalies in such subjects, and further incorporates and/or accesses an artificial intelligence neural network;
outputting said information and indications to the computer system;
incorporating said information and indications into the subject records and databases and inputting them to the neural network;
having the neural network match said information and indications against the databases, with the neural network generating match probabilities for various diagnoses, probabilities and suggestions for remedial action based on the likelihood of these various diagnoses, probabilities and suggestions for remedial action corresponding to the information and indications, and thereby determining the likeliest one or more diagnoses, probabilities and suggestions for remedial action; and
having the neural network output these diagnoses, probabilities and suggestions for remedial action.

2. The method of claim 1, further comprising having the diagnoses, probabilities and suggestions for remedial action that are output by the neural network also incorporated into the databases.

3. The method of claim 1, further comprising measuring the subject's vital signs and other machine-readable indications, and including the information derived from such measurement with the information outputted to the computer.

4. The method of claim 1, further comprising:
observation of the subject by skilled practitioners;
rendering of evaluation and diagnosis of the subject by said skilled practitioners; and
including such observations, evaluation and diagnosis along with the information and indications output to the computer system, incorporated into the subject records and databases and input to the neural network.

5. The method of claim 1, in which said computer system, in addition to incorporating and/or accessing databases on the familial, environmental and occupational factors relating to anomalies in such subjects, also incorporates and/or accesses databases on the treatment and remediation of such anomalies, such as medical dictionaries and other medical guides, and repair manuals.

6. An apparatus for automated differentiated diagnosis of anomalies in a subject, comprising:
a computer system which incorporates and/or accesses databases on the familial, environmental and occupational factors relating to anomalies in such subjects, and further incorporates and/or accesses an artificial intelligence neural network, into which computer system are fed information and indications about the subject—including the subject's individual, family and medical or other remedial history, plus physical, environmental, occupational and lifestyle information—acquired through interview and/or accessing subject records resident in said computer system, with said computer system incorporating said information and indications into the subject records and databases and inputting them to the neural network;
the neural network matching said information and indications against the databases, with the neural network generating match probabilities for various diagnoses, probabilities and suggestions for remedial action based on the likelihood of these various diagnoses, probabilities and suggestions for remedial action corresponding to the information and indications, and thereby determining the likeliest one or more diagnoses, probabilities and suggestions for remedial action; and
the neural network outputting these diagnoses, probabilities and suggestions for remedial action.

7. The apparatus of claim 6, further comprising having said computer system incorporate into the databases the diagnoses, probabilities and suggestions for remedial action that are output by the neural network.

8. The apparatus of claim 6, in which the initial information and indications about the subject further comprise measurements of the subject's vital signs and other machine-readable indications.

9. The apparatus of claim 6, in which the initial information and indications about the subject further comprise evaluation and diagnosis of the subject by skilled practitioners, rendered after observation of the subject by said skilled practitioners, with the computer system further including such observations, evaluation and diagnosis along with the information and indications output to the computer system, incorporated into the subject records and databases and input to the neural network.

10. The apparatus of claim 6, in which said computer system, in addition to incorporating and/or accessing databases on the familial, environmental and occupational factors relating to anomalies in such subjects, also incorporates and/or accesses databases on the treatment and remediation of such anomalies, such as medical dictionaries and other medical guides and repair manuals.
